SEQUENCE LISTING (2 sequences)

SEQ ID NO: 1 - DNA, 1966 bp, Homo sapiens
gggggcgtggccccgagaaggcggagacaagatggccgcccatagcgcttggaggacctaagaggcggtggccggggccacgccccgggcaggagggccgctctgtgcgcgcccgctctatgatgcttgcgcgcgtcccccgcgcgccgcgctgcgggcggggcgggtctccgggattccaagggctcggttacggaagaagcgcagcgccggctggggagggggctggatgcgcgcgcacccggggggaggccgctgctgcccggagcaggaggagggggagagtgcggcgggcggcagcggcgctggcggcgactccgccatagagcaggggggccagggcagcgcgctcgccccgtccccggtgagcggcgtgcgcagggaaggcgctcggggcggcggccgtggccgggggcggtggaagcaggcgggccggggcggcggcgtctgtggccgtggccggggccggggccgtggccggggacggggacggggccggggccggggccgcggccgtcccccgagtggcggcagcggccttggcggcgacggcggcggctgcggcggcggcggcagcggtggcggcggcgccccccggcgggagccggtccctttcccgtcggggagcgcggggccggggcccaggggaccccgggccacggagagcgggaagaggatggattgcccggccctcccccccggatggaagaaggaggaagtgatccgaaaatctgggctaagtgctggcaagagcgatgtctactacttcagtccaagtggtaagaagttcagaagcaagcctcagttggcaaggtacctgggaaatactgttgatctcagcagttttgacttcagaactggaaagatgatgcctagtaaattacagaagaacaaacagagactgcgaaacgatcctctcaatcaaaataagggtaaaccagacttgaatacaacattgccaattagacaaacagcatcaattttcaaacaaccggtaaccaaagtcacaaatcatcctagtaataaagtgaaatcagacccacaacgaatgaatgaacagccacgtcagcttttctgggagaagaggctacaaggacttagtgcatcagatgtaacagaacaaattataaaaaccatggaactacccaaaggtcttcaaggagttggtccaggtagcaatgatgagacccttttatctgctgttgccagtgctttgcacacaagctctgcgccaatcacagggcaagtctccgctgctgtggaaaagaaccctgctgtttggcttaacacatctcaacccctctgcaaagcttttattgtcacagatgaagacatcaggaaacaggaagagcgagtacagcaagtacgcaagaaattggaagaagcactgatggcagacatcttgtcgcgagctgctgatacagaagagatggatattgaaatggacagtggagatgaagcctaagaatatgatcaggtaactttcgaccgactttccccaagrgaaaattcctagaaattgaacaaaaatgtttccactggcttttgcctgtaagaaaaaaaatgtacccgagcacatagagctttttaatagcactaaccaatgcctttttagatgtatttttgatgtatatatctattattcaaaaaatcatgtttattttgagtcctaggacttaaaattagtcttttgtaatatcaagcaggaccctaagatgaagctgagcttttgatgccaggtgcaatctactggaaatgtagcacttacgtaaaacatttgtttcccccacagttttaataagaacagatcaggaattctaaataaatttcccagttaaagattattgtgacttcactgtatataaacatatttttatactttattgaaaggggacacctgtacattcttccatcatcactgtaaagacaaataaatgattatattcacaaaaaaaaaaaaaaaaaa

SEQ ID NO: 2 - DNA, 1411 bp, Homo sapiens
  Messenger RNA demethylase antisense
cgcatgcatgcataagcttgctcgagtctagatttttttttttttttgtctgtgaatataatcatttatttgtctttacagtgatgatggaagaatgtacaggtgtcccctttcaataaagtataaaaatatgtttatatacagtgaagtcacaataatctttaactgggaaatttatttagaattcctgatctgttcttattaaaactgtgggggaaacaaatgttttacgtaagtgctacatttccagtagattgcacctggcatcaaaagctcagcttcatcttagggtcctgcttgatattacaaaagactaattttaagtcctaggactcaaaataaacatgattttttgaataatagatatatacatcaaaaatacatctaaaaaggcattggttagtgctattaaaaagctctatgtgctcgggtacattttttttcttacaggcaaaagccagtggaaacatttttgttcaatttctaggaattttcycttggggaaagtcggtcgaaagttacctgatcatattcttaggcttcatctccactgtccatttcaatatccatctcttctgtatcagcagctcgcgacaagatgtctgccatcagtgcttcttccaatttcttgcgtacttgctgtactcgctcttcctgtttcctgatgtcttcatctgtgacaataaaagctttgcagaggggttgagatgtgttaagccaaacagcagggttcttttccacagcagcggagacttgccctgtgattggcgcagagcttgtgtgcaaagcactggcaacagcagataaaagggtctcatcattgctacctggaccaactccttgaagacctttgggtagttccatggtttttataatttgttctgttacatctgatgcactaagtccttgtagcctcttctcccagaaaagctgacgtggctgttcattcattcgttgtgggtctgatttcactttattactaggatgatttgtgactttggttaccggttgtttgaaaattgatgctgtttgtctaattggcaatgttgtattcaagtctggtttacccttattttgattgagaggatcgtttcgcagtctctgtttgttcttctgtaatttactaggcatcatctttccagttctgaagtcaaaactgctgagatcaacagtatttcccaggtaccttgccaactgaggcttgcttctgaacttcttaccacttggactgaagtagtagacatcgctcttgccagcacttagcccagattttcggatcacttcctccttcttccatccgggggggagggccgggcaatccatcctcttcccgctctccgtggcccggggtcccctgggccccggccccgcgctccccgacgggaaagggaccggctccgtcgacgcggcc

(19) United States
(12) Patent Application Publication
(10) Pub. No.: US 2006/0009403 A1
(43) Pub. Date: Jan. 12, 2006

(54) Dna demethylase antisense and chemotherapy combination

(75) Inventors: Pascal Bigey, Paris (FR); Marie-Agnes Ivanov, Champigny-Sur-Marne (FR); Daniel Scherman, Paris (FR)

Correspondence Address: HELLER EHRMAN WHITE & MCAULIFFE LLP, 1717 RHODE ISLAND AVE, NW, WASHINGTON, DC, 20036-3001, US

(73) Assignee: Centre National De La Recherche Scientifique (CNRS), Paris (FR)

(21) Appl. No.: 10/506,766

(22) PCT Filed: Mar. 5, 2003
(86) PCT No.: PCT/FR03/00705

§ 371 Date: Nov. 12, 2004

(30) Foreign Application Priority Data
Mar. 7, 2002 (FR) 02/02879

(51) Int. Cl. A61K 48/00; A61K 31/70

(52) U.S. Cl. 514/44

(57) ABSTRACT

The invention concerns a combination product comprising an antisense oligonucleotide of the gene encoding MBD2-demethylase and at least an agent used in antitumour chemotherapy, in particular bleomycin, for simultaneous, separate or prolonged use for treating proliferative and inflammatory diseases, in particular for cancer treatment.

The present invention relates to a combination product comprising an antisense of the gene encoding MBD2 demethylase and at least one agent used in antitumor chemotherapy, in particular bleomycin, for simultaneous, separate or prolonged use for treating proliferative and inflammatory diseases, in particular for treating cancer.

DNA methylation is an important epigenetic mechanism which regulates gene expression (1-4). One of the characteristics of cancer cells lies in an aberrant methylation scheme (5). Two contradictory changes in the methylation scheme have previously been documented, namely the hypermethylation of selected genes (6) and overall hypomethylation (7).

At the current time, it is not entirely known which mechanisms are responsible for the changes observed in DNA methylation. It is possible that these changes are a consequence of the deregulation of expression of the various components of the DNA methylation machinery (8). The DNA methylation machinery is made up of DNA-methyltransferase (9), of demethylases (10, (11) and (12) and of methylated DNA binding proteins (MBDs) which interpret the DNA methylation signal (13). A certain number of observations support the hypothesis that deregulation of the maintenance DNA-methyltransferase DNMT1 plays an important role in tumorigenesis (14), (15) and (16). An important question is therefore whether other components of the DNA methylation machinery are themselves also essential in nature for cell transformation (8) and (17).

It has been proposed that the hypermethylation of tumor suppressor genes would serve as a mechanism for silencing essential genes which inhibit various steps of tumorigenesis. The consequence of this hypermethylation will be to promote the process resulting in cell transformation (18). Methylated cytosines are specifically recognized by MBDs (13) and (19-21), which associate with corepressors such as Sin3A, recruit histone deacetylases for methylated genes (22-26) and can be found in known transcription repression complexes, such as Mi2 (27).

Mecp2, which is the most well-characterized member of the family, is probably not very important as regards the silencing of genes during transformation, since it is not expressed in cancerous cells (20). Other candidate proteins must be involved. A recently characterized methylated DNA binding protein, MBD2, is an interesting candidate for the reasons disclosed below.

First of all, the MBD2 cDNA has been cloned from a cancer cell line cDNA library (28), and it has been found that it is expressed in breast cancer samples and cell lines (29). Secondly, the protein is involved not only in suppression of the gene by a mechanism similar to that which is presented for Mecp2 (24) and (27), but it has also been found that it also carries a demethylase activity (28).

The demethylase activity has previously been purified from a human non-small cell lung carcinoma line A549 (12), and it was similarly found that transfection of the embryonic cell line P19 with the Ha-Ras protooncogene results in an increase in the demethylase activity (30). It is not impossible that an increased demethylase activity is associated with tumorigenesis, and that it could in part be responsible for the overall hypomethylation observed in cancer cells (17). Thus, Mbd2/demethylase could be part of the machinery involved in mediating or interpreting the two contradictory changes associated with the DNA methylation scheme in cancer cells, namely hypermethylation and hypomethylation.

Although this demethylase activity has been contested by certain groups (24), it has been shown that Mbd2b/demethylase obtained by recombination, expressed in a heterologous cell line SF-9, exhibits demethylase activity. In addition, the cotransfection of Mbd2b/demethylase and of methylated plasmids causes demethylation of these plasmids, and the forced expression of Mbd2b/demethylase in PANC-1 cells results in demethylation and in the induction of the endogenous MUC-2 promoter.

The present invention provides the elements demonstrating that Mbd2/demethylase is effectively expressed in cancer cells, and that it is essential to the growth of tumor cells in culture and in vivo.

No combination of gene therapy and of chemotherapy, consisting in combining an agent used in antitumor chemotherapy with a gene therapy based on an antisense of a gene involved in the level of DNA methylation, such as that of MBD2/demethylase, has been described in the state of the art.

Now, by combining a chemotherapy using bleomycin and the intratumor electrotransfer of a plasmid encoding the genetic antisense of the human DNA demethylase MBD2, a powerful synergistic effect in the treatment of tumors is obtained. The main advantage of the invention is therefore its surprising effectiveness since, if one considers the complete cure rate for tumors, it is 10% using gene therapy by electrotransfer of the MBD2 demethylase gene alone, and also 10% with bleomycin chemotherapy alone, and this rate increases to 40% using the combination of the two treatments: gene therapy and chemotherapy.

### DESCRIPTION

Thus, the present invention relates to a combination product comprising at least one antisense oligonucleotide of the gene encoding MBD2 demethylase and at least one agent used in antitumor chemotherapy, for simultaneous, separate or prolonged use intended for the treatment of proliferative and inflammatory diseases.

In a particular embodiment, the antisense of the gene encoding MBD2 demethylase comprises at least 15 consecutive nucleotides of the sequence SEQ ID No. 1 or of the sequence complementary thereto, or of SEQ ID No. 2.

SEQ ID No. 1 corresponds to the sequence described in GENEBANK under the accession number AF 072242 (Homo sapiens methyl-CpG binding protein MBD2 (MBD2) mRNA, complete cds).

SEQ ID No.1

gggggcgtggccccgagaaggcggagacaagatggccgcccatagcgctt
ggaggacctaagaggcggtggccggggccacgccccgggcaggagggccg
ctctgtgcgcgcccgctctatgatgcttgcgcgcgtcccccgcgcgccgc
gctgcgggcggggcgggtctccgggattccaagggctcggttacggaaga
agcgcagcgccggctggggagggggctggatgcgcgcgcacccgggggga
ggccgctgctgcccggagcaggaggagggggagagtgcggcgggcggcag
cggcgctggcggcgactccgccatagagcaggggggccagggcagcgcgc
tcgccccgtccccggtgagcggcgtgcgcagggaaggcgctcggggcggc
ggccgtggccgggggcggtggaagcaggcgggccggggcggcggcgtctg
tggccgtggccggggccggggccgtggccggggacggggacggggccggg
gccggggccgcggccgtcccccgagtggcggcagcggccttggcggcgac
ggcggcggctgcggcggcggcggcagcggtggcggcggcgccccccggcg
ggagccggtccctttcccgtcggggagcgcggggccggggcccaggggac
cccgggccacggagagcgggaagaggatggattgcccggccctccccccc
ggatggaagaaggaggaagtgatccgaaaatctgggctaagtgctggcaa
gagcgatgtctactacttcagtccaagtggtaagaagttcagaagcaagc
ctcagttggcaaggtacctgggaaatactgttgatctcagcagttttgac
ttcagaactggaaagatgatgcctagtaaattacagaagaacaaacagag
actgcgaaacgatcctctcaatcaaaataagggtaaaccagacttgaata
caacattgccaattagacaaacagcatcaattttcaaacaaccggtaacc
aaagtcacaaatcatcctagtaataaagtgaaatcagacccacaacgaat
gaatgaacagccacgtcagcttttctgggagaagaggctacaaggactta
gtgcatcagatgtaacagaacaaattataaaaaccatggaactacccaaa
ggtcttcaaggagttggtccaggtagcaatgatgagacccttttatctgc
tgttgccagtgctttgcacacaagctctgcgccaatcacagggcaagtct
ccgctgctgtggaaaagaaccctgctgtttggcttaacacatctcaaccc
ctctgcaaagcttttattgtcacagatgaagacatcaggaaacaggaaga
gcgagtacagcaagtacgcaagaaattggaagaagcactgatggcagaca
tcttgtcgcgagctgctgatacagaagagatggatattgaaatggacagt
ggagatgaagcctaagaatatgatcaggtaactttcgaccgactttcccc
aagrgaaaattcctagaaattgaacaaaaatgtttccactggcttttgcc
tgtaagaaaaaaaatgtacccgagcacatagagctttttaatagcactaa
ccaatgcctttttagatgtatttttgatgtatatatctattattcaaaaa
atcatgtttattttgagtcctaggacttaaaattagtcttttgtaatatc
aagcaggaccctaagatgaagctgagcttttgatgccaggtgcaatctac
tggaaatgtagcacttacgtaaaacatttgtttcccccacagttttaata
agaacagatcaggaattctaaataaatttcccagttaaagattattgtga
cttcactgtatataaacatatttttatactttattgaaaggggacacctg
tacattcttccatcatcactgtaaagacaaataaatgattatattcacaa
aaaaaaaaaaaaaaaa

Among the preferred antisense sequences of the invention, more particularly noted is the sequence SEQ ID No. 2, which corresponds to the complete messenger RNA of the demethylase in the antisense orientation:

cgcatgcatgcataagcttgctcgagtctagatttttttttttttttgtc
tgtgaatataatcatttatttgtctttacagtgatgatggaagaatgtac
aggtgtcccctttcaataaagtataaaaatatgtttatatacagtgaagt
cacaataatctttaactgggaaatttatttagaattcctgatctgttctt
attaaaactgtgggggaaaacaaatgtttttacgtaagtgctacatttcc
agtagattgcacctggcatcaaaagctcagcttcatcttagggtcctgct
tgatattacaaaagactaattttaagtcctaggactcaaaataaacatga
ttttttgaataatagatatatacatcaaaaatacatctaaaaaggcattg
gttagtgctattaaaaagctctatgtgctcgggtacattttttttcttac
aggcaaaagccagtggaaacatttttgttcaatttctaggaattttcyct
tggggaaagtcggtcgaaagttacctgatcatattcttaggcttcatctc
cactgtccatttcaatatccatctcttctgtatcagcagctcgcgacaag
atgtctgccatcagtgcttcttccaatttcttgcgtacttgctgtactcg
ctcttcctgtttcctgatgtcttcatctgtgacaataaaagctttgcaga
ggggttgagatgtgttaagccaaacagcagggttcttttccacagcagcg
gagacttgccctgtgattggcgcagagcttgtgtgcaaagcactggcaac
agcagataaaagggtctcatcattgctacctggaccaactccttgaagac
ctttgggtagttccatggtttttataatttgttctgttacatctgatgca
ctaagtcctgtagcctcttctcccaqgaaaagctgacgtggctgttcatt
cattcgttgtgggtctgatttcactttattactaggatgatttgtgactt
tggttaccggttgtttgaaaattgatgctgtttgtctaattggcaatgtt
gtattcaagtctggtttacccttattttgattgagaggatcgtttcgcag
tctctgtttgttcttctgtaatttactaggcatcatctttccagttctga
agtcaaaactgctgagatcaacagtatttcccaggtaccttgccaactga
ggcttgcttctgaacttcttaccacttggactgaagtagtagacatcgct
cttgccagcacttagcccagattttcggatcacttcctccttcttccatc
cggggggggagggccgggcaatccatcctcttcccgctctccgtggcccg
gggtcccctgggccccggccccgcgctccccgacgggaaagggaccggct
ccgtcgacgcggcc

This antisense sequence was used in the context of the experiments presented in Example 1.

Thus, the invention is directed toward a combination product as mentioned above, in which the antisense comprises at least:
a) 15 consecutive nucleotides of the sequence SEQ ID No. 1 or of the sequence complementary thereto, or of the sequence SEQ ID No. 2, or
b) a sequence capable of hybridizing selectively with one of the sequences defined in a).

The expression “sequence capable of hybridizing selectively” is intended to mean the sequences which hybridize with the abovementioned sequences at a level significantly greater than the background noise. The background noise may be related to the hybridization of other DNA sequences as are present, in particular other mRNAs that are present in the targeted tumor cells. The level of the signal generated by the interaction between the sequence capable of hybridizing selectively and the sequences defined by SEQ ID Nos. 1 and 2 above is generally 10 times, preferably 100 times, more intense than that of the interaction of the other DNA sequences generating the background noise. The level of interaction can be measured, for example, by labeling the sequence used as a probe with radioactive elements, such as ³²P. The selective hybridization is generally obtained by using very strict medium conditions (for example 0.03M NaCl and 0.03M sodium citrate at approximately 50° C.-60° C.). The hybridization can be carried out according to the usual methods of the state of the art (in particular Sambrook et al., 1989, Molecular Cloning: A Laboratory Manual).

The expression “agent used in antitumor chemotherapy” is intended to denote antineoplastic agents. Among these agents, mention may be made of:
the compounds belonging to the bleomycin family (Mueller et al., Cancer, Vol. 40, p. 2787 (1977), Umezawa et al., Journal of Antibiotics, 19A, p. 210 (1966), U.S. Pat. No. 4,472,304, FR2530639, and U.S. Pat. No. 3,922,262), in particular bleomycin,
the various cytolytic agents such as dacarbazine, hydroxycarbamide, asparaginase, mitoguazone and plicamycin,
the methylating agents, such as streptozotocin (2-deoxy-2-(3-methyl-3-nitrosoureido)-D-glucopyranose), procarbazine (N-(1-methylethyl)-4-[(2-methylhydrazino)methyl]benzamide), dacarbazine or DTIC (5-(3,3-dimethyl-1-triazenyl)-1H-imidazole-4-carboxamide), and temozolomide (8-carbamoyl-3-methylimidazo[5.1-d]-1,2,3,5-tetrazin-4-(3H)-one,
the chloroethylating agents, such as HECNU (1-(2-chloroethyl)-3-(2-hydroxyethyl)-1-nitrosourea), BCNU (1,3-bis(2-chloroethyl)-1-nitrosourea or carmustine, Bristol-Meyers), ACNU (1-(2-chloro-ethyl)-3-(4-amino-2-methyl-5-pyrimidinyl)methyl-1-nitrosourea), CCNU (1-(2-chloroethyl)-3-cyclo-hexyl-1-nitrosourea or lomustine), MeCCNU (1-(2-chloroethyl)-3-(4-methylcyclohexyl)-1-nitrosourea or semustine), fotemustine (1-[N-(2-chloroethyl)-N-nitrosoureido]ethylphosphonic acid diethyl ester) and clomesone (2-chloroethylmethylsulfonyl-methanesulfonate) (Pegg et al., Prog. Nucleic Acid Research Molec. Biol. 51: 167-223 (1995)). These agents are further described in Colvin and Chabner, Alkylating Agents. In: Cancer,
other alkylating compounds such as agents of the type Ecteinascidin 743, and the duocarmycins (Boger et al. J. Org. Chem. 1990, 55, 4499; Boger et al. J. Am. Chem. Soc. 1990, 112, 8961; Boger et al. J. Am. Chem. Soc. 1991, 113, 6645; Boger et al. J. Am. Chem. Soc. 1993, 115, 9872; Boger et al. Bioorg. Med. Chem. Lett. 1992, 2, 759),
the pro-apoptotic agents selected from glucocorticoid derivatives, topoisomerase inhibitors such as topoisomerase 2 inhibitors, for example anthracyclines, epipodophyllotoxin, such as etoposide, topoisomerase 1 inhibitors, for example camptothecin derivatives,
the antimetabolites such as antifolates, for example methotrexate, antipurines, for example 6-mercaptopurine, antipyrimidines, for example 5-fluorouracil,
from the antimitotics such as the vinca-alkaloids, taxoids such as taxotere.

These antineoplastic agents are described in Actualité Pharmaceutiques [Pharmaceutical News] No. 302 (October 1992), pages 38 to 39, and 41 to 43.

In a preferred aspect, the invention is directed toward a combination product as defined above, in which the agent is selected from compounds belonging to the bleomycin family, in particular bleomycin.

In another particular embodiment, the invention relates to a combination product mentioned above, in which the antisense oligonucleotide of the gene encoding MBD2 demethylase is carried by a vector comprising a promoter which allows its effective expression in a eukaryotic cell. This vector may also comprise a poly A transcription termination sequence.

Preferably, the vector consists of a plasmid. In fact, the use of a plasmid is more economical and safer than the use of viruses. In addition, this embodiment of the invention allows readministration without triggering an immune response. This plasmid advantageously comprises a promoter, the antisense sequence according to the invention and a transcription terminating sequence. Preferably, the sequence of the antisense is inserted into the plasmid pcDNA3.1HisA from the company InVitrogen.

The product according to the invention may also comprise one or more pharmaceutically acceptable vehicle(s). It is intended in particular for simultaneous, separate or prolonged use intended for the treatment of cancer.

In this sense, in a preferred embodiment, the formulations are suitable for administration by intratumor injection.

The techniques for transferring the plasmid into the target cells are well known to those skilled in the art. In particular, reference will be made to the techniques for electrotransfer into eukaryotic cells described in WO 99/01157 and Bettan et al., Bioelectrochemistry and Bioenergetics, 2000, 52:83-90. In WO 99/01157, a method for in vivo transfer of nucleic acids is proposed using weak electric fields between 1 and 600 V/cm. Other approaches are described in Wolf et al., Science 247, 1465-68, 1990; and Davis et al., Proc. Natl. Acad. Sci. USA 93, 7213-18, 1996), in which the DNA is associated with compounds intended to promote its transfection, such as proteins, liposomes, charged lipids or cationic polymers, such as polyethyleneimine, which are good in vitro transfecting agents (Behr et al., Proc. Natl. Acad. Sci. USA 86, 6982-6, 1989; Felgner et al., Proc. Natl. Acad. Sci. USA 84, 7413-7, 1987; Boussif et al., Proc. Natl. Acad. Sci. USA 92, 7297-301, 1995).

Thus, in accordance with the invention, the antisense can also be transferred in the form of double-stranded DNA or of a plasmid as mentioned above, possibly in combination with a molecule which promotes the transfer and/or using a weak electric field.

The invention also extends to any application for treating cancer, comprising the use of a combination product mentioned above and a third active substance used in the context of the treatment of the cancer. In this respect, the invention covers a tritherapy comprising the administration of the combination product according to the invention and a third active substance.

Mbd2/demethylase is expressed in tumors in vivo and is overexpressed in a significant percentage of tumors in a manner similar to Dmnt1. Although our analysis of a limited number of tumors does not prove that Mbd2/demethylase is generally deregulated in cancer cells, our data are compatible with this model. Secondly, we show that the antisense-mediated inhibition of Mbd2/demethylase results in changes in genomic methylation and in an inhibition of tumorigenesis in vitro. Various methods of antisense expression have been used in order to exclude the possibility that the changes observed reflect a certain idiosyncratic property of the vector. Transient expression of the antisense is sufficient to inhibit the anchorage- and contact-inhibited growth, which indicates that Mbd2/demethylase is necessary for maintaining the transformed state, and that its inhibition has immediate effects on the growth of cancer cells.

Similarly, the introduction of a vector expressing the antisense of Mbd2/demethylase into human tumors, that had been passed in nude mice in the form of xenographs, resulted in a decrease in the growth of the tumor, which shows that Mbd2/demethylase is necessary for maintaining the transformed state. Whereas the expression of the Mbd2/demethylase antisense considerably inhibits tumorigenesis in vitro, it has a limited effect on tumors in vivo. This could reflect the difficulty that exists in effectively delivering and expressing the antisense vectors in all the cells of a tumor in vivo, rather than an indication of the limited impact of the inhibition of the target.

Since Mbd2/demethylase can either repress or demethylate methylated genes, it is possible for a certain number of genes to be affected by one or other of these processes. Inhibition of the repression, mediated by Mbd2/demethylase, of the activity of methylated genes could result in an activation of a certain number of tumor suppressors. Moreover, the demethylase activity could be required for inhibiting an aberrant methylation of genes which are essential for the transformed phenotype. Inhibition of the demethylase could result in an ectopic methylation, essential genes being silenced stochastically.

Since the two activities of Mbd2/demethylase must affect a wide range of genes, a possible result could have been a collapse of the gene expression program. Such a possibility would have to have limited the therapeutic potential of the inhibition of Mbd2/demethylase. However, analysis of the gene scheme of the cells in which Mbd2/demethylase is inhibited does not support this hypothesis.

Thus, the inhibition of Mbd2/demethylase results in a repression and in an induction of the expression of the genes involved in the tumoral process, but does not present any disadvantage for a therapeutic application. Changes in gene expression after treatment with the Mbd2/demethylase antisense appear to be limited, however these changes, strengthened by an alkylating agent, are responsible for the strong inhibition of tumorigenesis in vitro.

Thus, the invention proposes the joint use of Mbd2/demethylase as an anticancer target, and a DNA alkylating agent. The fact that the cell cycle of normal cells is not affected by this treatment, and the fact that this treatment does not cause any massive changes in gene expression, increase the advantage of this target. The inhibition of Mbd2/demethylase could have a therapeutic effect on two levels, one in re-establishing the normal state of genomic methylation by inhibition of a demethylase that is undergoing aberrant overregulation, and another in preventing that which causes incorrectly methylated tumor suppressor genes to become silent, which genes are essential to maintaining an appropriate regulation of cell growth.

### EXAMPLE 1

### Combination of Gene Therapy (Intratumor Electrotransfer of Plasmids Encoding the DNA Demethylase Antisense) and of Chemotherapy (Intramuscular Injection of Bleomycin)

Two series of experiments were carried out in nude mice weighing 18 to 20 g. The mice were implanted on one side with H1299 tumor grafts (human non-small cell lung tumors) of approximately 20 mm³. The tumors developed, to reach a volume of 20 to 150 mm³. The mice were sorted as a function of the size of the tumors and were divided up into homogeneous batches reaching tumor volumes of 50 to 80 mm³ (n=10 to 13). The mice were anesthetized with a mixture of ketamine and xylazine.

1.1 Experiment 1: Effect on Tumor Growth

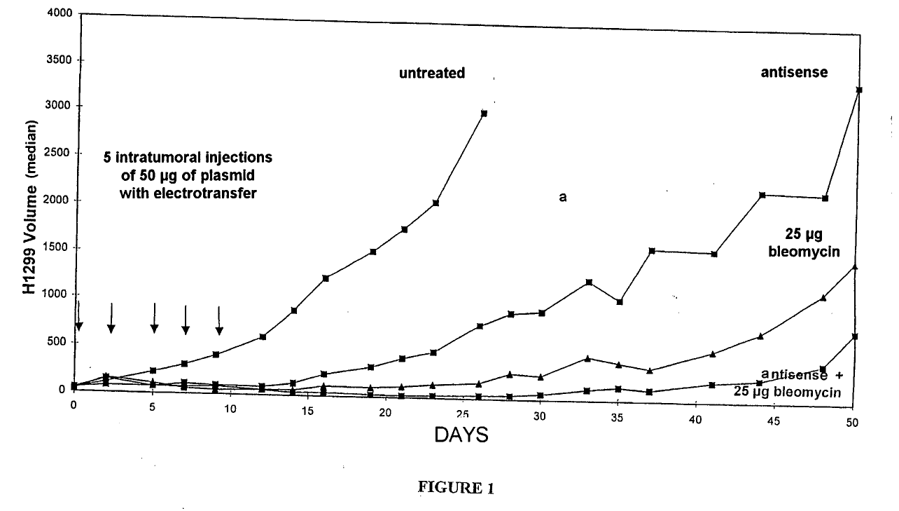

The results are illustrated in FIG. 1 and the statistical analysis is given in table 1 below.

TABLE 1
STATISTICAL ANALYSIS
Experiment 1

| | Day 1000 mm³ |
|---|---|
| | (median) # |
| Group 1: untreated tumors | 14.50 |
| Group 3: 25 μg bleomycin | 44.40 |
| Group 4: DNA demethylase | 29.10 |
| antisense | |
| Group 6: DNA demethylase | 52.01 |
| antisense + 25 μg bleomycin | |

| | | Log-Rank | | |
|---|---|---|---|---|
| | Student's t | Kaplan-Meier | | |
| | test | Risk of reaching | | |
| | Mean | 1000 mm³ of tumor | | |
| Statistical comparison | comparison | volume | | |
| DNA demethylase antisense | p < 0.0001 | *** | p < 0.0001 | *** |
| versus untreated | | | | |
| 25 μg bleomycin versus | p < 0.0001 | *** | p < 0.0001 | *** |
| untreated | | | | |
| DNA demethylase antisense + | p = 0.1079 | NS | p = 0.1946 | NS |
| 25 μg bleomycin versus | | | | |
| 25 μg bleomycin | | | | |
| DNA demethylase antisense + | p < 0.0001 | *** | p < 0.0001 | *** |
| 25 μg bleomycin versus | | | | |
| untreated | | | | |

1.1.1 Control Tumors:

A series of tumors was subjected to no treatment.

1.1.2 Tumors Treated with the Gene Encoding the DNA Demethylase Antisense, Alone:

Five electrotransfers of 50 μg of plasmid in 80 μl of 150 mM NaCl were carried out in the tumors on the days indicated by the arrows. The plasmid solution was injected longitudinally at the periphery of the tumor using a Hamilton syringe. The lateral faces of the tumors were coated with conducting gel and the tumors were placed between 2 flat stainless steel electrodes 0.4 to 0.7 cm apart. Twenty to 30 seconds after the injection, the plasmids were electrotransferred using a commercial (square) electrical pulse generator (Jouan Electropulser PS 15). Each tumor was subjected to 500 V/cm delivered in 8 pulses lasting 20 msec at a frequency of 1 Hertz.

1.1.3 Tumors Treated with Bleomycin Alone:

Twenty-five μg of bleomycin/animal in 50 μl of 150 mM NaCl were injected bilaterally into the tibialis cranialis muscle and, 30 minutes later, each tumor was subjected to 1 electrotransfer as explained above.

1.1.4 Tumors Treated with a Combination of the 2 Treatments (Antisense and Bleomycin):

Twenty-five μg of bleomycin/animal in 50 μl of 150 mM NaCl were injected bilaterally into the tibialis cranialis muscle and, 30 minutes later, 50 μg of antisense plasmid in 80 μl of 150 mM NaCl were injected and electrotransferred. Four other electrotransfers of 50 μg of antisense plasmid in 80 μl of 150 mM NaCl were subsequently carried out in the tumors on the days indicated by the arrows.

The tumor volumes were measured individually for each tumor using an electronic slide gauge with a digital display, according to the formula (length×width×thickness)/2.

The median of the tumor volumes was reported in the form of a graph, as a function of time.

1.2 Experiment 2: Effect on Tumor Growth

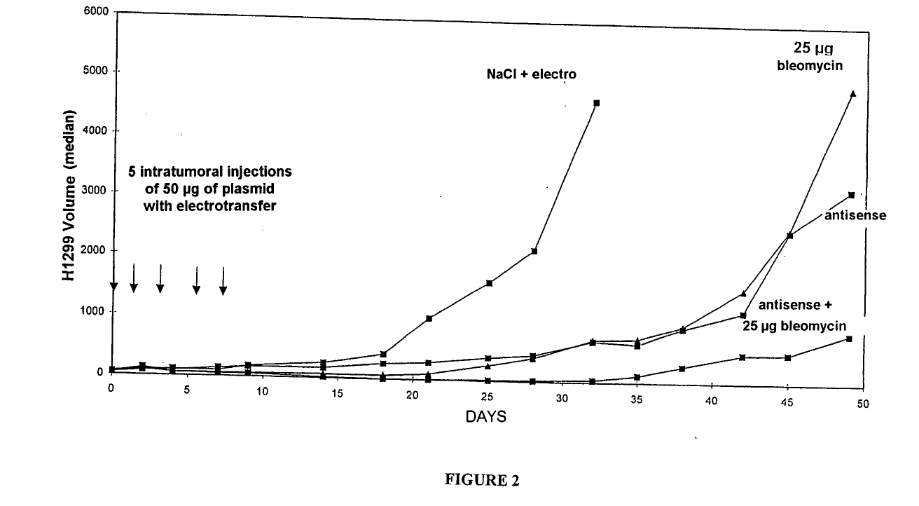

The results are illustrated in FIG. 2 and the statistical analysis is given in table 2 below.

TABLE 2
STATISTICAL ANALYSIS
Experiment 2

| | D 1000 mm³ | | | |
|---|---|---|---|---|
| | (median) # | | | |
| Group 1: NaCl/ET | 20.90 | | | |
| Group 2: 25 μg bleomycin | 38.00 | | | |
| Group 3: DNA demethylase | 38.60 | | | |
| antisense | | | | |
| Group 4: DNA demethylase | 52.00 | | | |
| antisense + 25 μg bleomycin | | | | |
| | | Log-Rank | | |
| | Student's | Kaplan-Meier | | |
| | t test | Risk of reaching | | |
| | Mean | 1000 mm³ of tumor | | |
| Statistical comparison | comparison | volume | | |
| DNA demethylase antisense | p = 0.0201 | * | p = 0.0029 | ** |
| versus NaCl/ET | | | | |
| 25 μg bleomycin versus | p = 0.0008 | *** | p = 0.0001 | *** |
| NaCl/ET | | | | |
| DNA demethylase antisense + | p = 0.0088 | ** | p = 0.0056 | ** |
| 25 μg bleomycin versus | | | | |
| 25 μg bleomycin | | | | |
| DNA demethylase antisense + | p = 0.0001 | *** | p < 0.0001 | *** |
| 25 μg bleomycin/NaCl/ET | | | | |

# number of days to reach 1000 mm³ of tumor volume

1.2.1 Control Tumors:

Five electrotransfers of 80 μl of 150 mM NaCl were carried out in the tumors on the days indicated by the arrows.

1.2.2 Tumors Treated with the Gene Encoding the DNA Demethylase Antisense, Alone

Fifty μl of 150 mM NaCl were injected bilaterally into the tibialis cranialis muscle and, 30 minutes later, an electrotransfer of 50 μg of antisense plasmid in 80 μl of 150 mM NaCl was carried out. Four other electrotransfers of 50 μg of antisense plasmid in 80 μl of 150 mM NaCl were subsequently carried out in the tumors on the days indicated by the arrows.

1.2.3 Tumors Treated with Bleomycin Alone:

Twenty-five μg of bleomycin/animal in 50 μl of 150 mM NaCl were injected bilaterally into the tibialis cranialis muscle and, 30 minutes later, each tumor was injected with 80 μl of 150 mM NaCl and subjected to an electrotransfer. Four other electrotransfers of 80 μl of 150 mM NaCl were subsequently carried out in the tumors on the days indicated by the arrows.

1.2.4 Tumors Treated with a Combination of the 2 Treatments (Antisense and Bleomycin):

Twenty-five μg of bleomycin/animal in 50-μl of 150 mM NaCl were injected bilaterally in the tibialis cranialis muscle and, 30 minutes later, an electrotransfer of 50 μg of antisense plasmid in 80 μl of 150 mM NaCl was carried out. Four other electrotransfers of 50 μg of antisense plasmid in 80 μl of 150 mM NaCl were subsequently carried out in the tumors on the days indicated by the arrows.

The tumor volumes were measured individually for each tumor using an electronic slide gauge with a digital display, according to the formula (length×width×thickness)/2.

The median of the tumor volumes was reported in the form of a graph, as a function of time.

1.3 Results and Conclusion

The combination of gene therapy with the gene encoding the human DNA demethylase antisense and of chemotherapy with bleomycin makes it possible to induce a cumulative delay of 31 to 38 days in the growth of H1299 tumors.

Such a delay in tumor growth was never achieved with the treatments administered separately, such as the gene therapy alone (15 to 18 days) or the chemotherapy alone (17 to 30 days) (table 3 below).

TABLE 3
Combination of gene therapy and of chemotherapy
Effect of multiple intratumor electrotransfers of plasmids
encoding the human DNA demethylase antisense, combined with
a treatment with bleomycin, on the growth of H1299 tumors
a) Delay in tumor growth

| | Experiment 1 | | Experiment 2 | |
|---|---|---|---|---|
| | | Delay in | | Delay in |
| | | growth | | growth |
| | | Treatment | | Treatment |
| | D1000 | versus | D1000 | versus |
| | * | untreated | * | electro/NaCl |
| Untreated | D14 | | | |
| ET/NaCl | | | D21 | |
| Demethylase antisense | D29 | 15 days | D39 | 18 days |
| 25 μg bleomycin | D44 | 30 days | D38 | 17 days |
| Demethylase | D52 | 38 days | D52 | 31 days |
| antisense/25 μg | | | | |
| bleomycin | | | | |

D1000* = number of days required to reach a tumor volume of 1000 mm³

The combination of the gene therapy and the chemotherapy induces a synergistic effect on the tumor cure rate, since a tumor cure rate of 30 to 40% was obtained with the combined treatment, compared with 10% only with the treatments administered separately (table 4 below).

TABLE 4
Combination of gene therapy and of chemotherapy
b) Tumor cure rate

| | Experiment 1 | Experiment 2 |
|---|---|---|
| | Number of tumors | Number of tumors |
| | cured | cured |
| Untreated | 0/11 | |
| NaCl/electro | | 011 |
| Demethylase | 0/13 | 1/10 |
| antisense | | D53 |
| 25 μg bleomycin | 1/13 | 1/11 |
| | D54 | D53 |
| Demethylase | 3/11 | 4/10 |
| antisense/25 μg | D33/D69/D69 | D32/D35/D53/D53 |
| bleomycin | | |

Rem: the tumors cured are tumors which are no longer measurable
Dx: absence of tumors up to the day indicated, beyond which the mouse died

### REFERENCES

1. Razin, A. & Szyf, M. (1984) Biochim Biophys Acta 782, 331-42.
2. Razin. A. & Cedar, H. (1977) Proc Natl Acad Sci USA 74, 2725-8.
3. Razin, A. & Riggs, A. D. (1980) Science 210, 604-10.
4. Razin, A. (1998) Embo J 17, 4905-8.
5. Baylin, S. B., Herman J. G., Graff, J. R., Vertino, P. M. & Issa, J. P. (1998) Adv Cancer Res 72, 141-96.
6. Baylin, S. B. (1992) AIDS Res Hum Retroviruses 8, 811-20.
7. Feinberg, A. P. & Vogelstein, B. (1983) Nature 301, 89-92.
8. Szyf, M. (1994) Trends Pharmacol Sci 15, 233-8.
9. Robertson, K. D., Uzvolgyi, E., Liang, G., Talmadge, C., Sumegi, J., Gonzales, F. A. & Jones, P. A. (1999) Nucleic Acids Res 27, 2291-8.
10. Weiss, A. & Cedar, H. (1997) Genes Cells 2, 481-6.
11. Jost, J. P., Siegmann, M., Sun, L. & Leung, R. (1995) J Biol Chem 270, 9734-9.
12. Ramchandani, S., Bhattacharya, S. K., Cervoni, N. & Szyf, M. (1999) Proc Natl Acad Sci USA 96, 6107-12.
13. Hendrich, B. & Bird, A. (1998) Mol Cell Biol 18, 6538-47.
14. MacLeod, A. R. & Szyf, M. (1995) J Biol Chem 270, 8037-43.
15. Laird, P. W., Jackson-Grusby, L., Fazeli, A., Dickinson, S. L., Jung, W. E., Li, E., Weinberg, R. A. & Jaenisch, R. (1995) Cell 81, 197-205.
16. Ramchandani, S., MacLeod, A. R., Pinard, M., von Hofe, E. & Szyf, M. (1997) Proc Natl Acad Sci USA 94, 684-9.
17. Szyf, M. (1998) Cancer Metastasis Rev 17, 219-31.
18. Baylin, S. B. & Herman, J. G. (2000) Trends Genet 16, 168-74.
19. Meehan, R. R., Lewis, J. D. & Bird, A. P. (1992) Nucleic Acids Res 20, 5085-92.
20. Lewis, J. D., Meehan, R. R., Henzel, W. J., Maurer-Fogy, I., Jeppesen, P., Klein, F. & Bird, A. (1992) Cell 69, 905-14.
21. Cross, S. H., Meehan, R. R., Nan, X. & Bird, A. (1997) Nat Genet 16, 256-9.
22. Nan, X., Ng, H. H., Johnson, C. A., Laherty, C. D., Turner, B. M., Eisenman, R. N. & Bird, A. (1998) Nature 393, 386-9.
23. Jones, P. L. Veenstra, G. J., Wade, P. A., Vermaak, D., Kass, S. U., Landsberger, N., Strouboulis, J. & Wolffe, A. P. (1998) Nat Genet 19, 187-91.
24. Ng, H. H., Zhang, Y., Hendrich, B., Johnson, C. A., Turner, B. M., Erdjument-Bromage, H. Tempst, P., Reinberg, D. & Bird, A. (1999) Nat Genet 23, 58-61.
25. Ng, H. H., Jeppesen, P. & Bird, A. (2000) Mol Cell Biol 20, 1394-406.
26. Boeke, J., Ammerpohl. O. Kegel, S., Moehren, U. & Renkawitz. R. (2000) J Biol. Chem.

27. Wade, P. A., Gegonne, A., Jones, P. L., Ballestar, E., Aubry, F. & Wolffe, A. P. (1999) Nat Genet 23, 62-6.
28. Bhattacharya, S. K., Ramchandani, S., Cervoni, N. & Szyf, M. (1999) Nature 397, 579-83.
29. Vilain, A., Vogt, N. Dutrillaux, B. & Malfoy, B. (1999) FEBS Lett 460, 231-4.
30. Szyf, M., Theberge, J. & Bozovic, V. (1995) J Biol Chem 270, 12690-6.

1. A combination product comprising at least one antisense oligonucleotide of the gene encoding MBD2 demethylase and at least one agent used in antitumor chemotherapy, for simultaneous, separate or prolonged use intended for the treatment of proliferative and inflammatory diseases.

2. The combination product of claim 1, wherein the antisense of the gene encoding MBD2 demethylase comprises at least:
a) 15 consecutive nucleotides of the sequence SEQ ID No. 1 or of the sequence complementary thereto, or of the sequence SEQ ID No. 2, or
b) a sequence capable of hybridizing selectively with one of the sequences defined in a).

3. The combination product of one of claim 1 or 2, wherein the agent used in antitumor chemotherapy is a compound belonging to the bleomycin family.

4. The combination product of one of claim 1 or 2, wherein the agent used in antitumor chemotherapy is an antineoplastic agent capable of methylating DNA.

5. The combination product of one of claim 1 or 2, wherein the agent used in antitumor chemotherapy is a chloroethylating agent.

6. The combination product of one of claim 1 or 2, wherein the agent used in antitumor chemotherapy is selected from the group consisting of:
a cytolytic,
a pro-apoptotic agent,
an antimetabolite, and
an antimitotic.

7. The combination product of claim 1, wherein the antisense oligonucleotide of the gene encoding MBD2 demethylase is in a vector comprising a promoter which allows its effective expression in a eukaryotic cell.

8. The combination product of claim 7, which further comprises a poly A transcription termination sequence.

9. The combination product of claim 7, wherein the vector is a plasmid.

10. The combination product of claim 1, wherein the antisense oligonucleotide is a double-stranded DNA.

11. The combination product of claim 10, which further comprises one or more elements which promote the transfer of the antisense oligonucleotide into the target cells.

12. The combination product of claim 11, wherein the antisense oligonucleotide is suitable for administration in vivo by electrotransfer.

13. The combination product of claim 12, further comprising one or more pharmaceutically acceptable vehicle(s).

14. The combination product of claim 13, for simultaneous, separate or prolonged use in the treatment of cancer.

15. The combination product of claim 14, which is suitable for administration by intratumor injection.

16. The combination product of claim 3, wherein said compound is bleomycin.

17. The combination product claim 4, wherein said agent is selected from the group consisting of streptozotocin, procarbazine, dacarbazine and temozolomide.

18. The combination product of claim 5, wherein said agent is selected from the group consisting of chloroethylating agent 1-(2-chloroethyl)-3-(2-hydroxyethyl)-1-nitrosourea,
1-(chloroethyl)-3-(2-hydroxyethl)-1-nitrosourea,
1,3-bis(2-chloroethyl)-1-nitrosourea,
1-(2-chloroethyl)-3-(4-amino-2-methyl-5-pyrimidinyl)methyl 1-nitrosourea,
1-(2-chloroethyl)-3-cyclohexyl-1-nitrosourea,
1-(2-chloroethyl)-3-(4-methylcyclohexyl)-1-nitrosourea,
1-[N-(2-chloroethyl)-N-nitrosoureido]ethylphosphonic acid diethyl ester, and
2-chloroethylmethylsulfonylmethanesulfonate.

19. The combination product of claim 6, wherein said cytolytic agent is selected from the group consisting of dacarbazine, hydroxycarbamide, asparaginase, mitoguazone and plicamycin.

20. The combination product of claim 6, wherein said pro-apoptotic agent is selected from the group consisting of glucocorticoid derivatives, topoisomerase 2 inhibitors and topoisomerase 1 inhibitors.

21. The combination product of claim 20, wherein said topoisomerase 2 inhibitor is an anthracycline epipodophyllotoxin.

22. The combination product of claim 21, wherein said antracycline epipodophyllotoxin is etoposide.

23. The combination product of claim 20, wherein said topoisomerase 1 inhibitor is a camptothecin derivative.

24. The combination product of claim 6, wherein said antimetabolite agent is selected from antifolates, the group consisting of antipurines, and antipyrimidines.

25. The combination product of claim 24, wherein said antifolate is methotrexate.

26. The combination product of claim 24, wherein said antipurine is 6-mercaptopurine.

27. The combination product of claim 24, wherein said antipyrimidine is 5-fluorouracil.

28. The combination product of claim 6, wherein said antimitotic agent is selected from the group consisting of vincaalkaloids and taxoids.

29. The combination product of claim 12, wherein the electro transfer is by weak electric fields of between 1 and 600 V/cm.
